(19) United States
(12) Patent Application Publication
(10) Pub. No.: US 2008/0193914 A1
(43) Pub. Date: Aug. 14, 2008

(54) Biomarker derivation method

(76) Inventor: Randall Thomason, Suprise, AZ (US)

Correspondence Address: ROBERT A. PARSONS, 4000 N. CENTRAL AVENUE, SUITE 1220, PHOENIX, AZ, 85012, US

(21) Appl. No.: 11/697,621

(22) Filed: Apr. 6, 2007

Related U.S. Application Data
(60) Provisional application No. 60/744,361, filed on Apr. 6, 2006.

(51) Int. Cl. C12Q 1/00

(52) U.S. Cl. 435/4

(57) ABSTRACT

A method for deriving a quantitative value for a specific biomarker of an individual includes providing a set of biomarkers from a blood sample of the individual, the set of biomarkers not including the specific biomarker. Adjusted marker values for biomarkers not present in the set of biomarkers are provided. Algorithmic manipulation of at least a portion of the set of biomarkers and the adjusted marker values with a precise numerical value is then used to derive the quantitative value for the specific biomarker.

### CROSS-REFERENCE TO RELATED APPLICATIONS

This application claims the benefit of U.S. Provisional Application No. 60/744,361, filed 6 Apr. 2006.

### FIELD OF THE INVENTION

This invention relates to medical diagnostics.

More particularly, the present invention relates to employing blood chemistry analysis to determine values for biomedical markers.

### BACKGROUND OF THE INVENTION

In the field of medical diagnostics through blood chemistry, various biochemical markers (biomarkers) are used to assist in determining a condition in a patient. In conventional “check-ups” a basic blood chemistry panel (i.e. comprehensive metabolic panel) and a complete blood count (CBC) are typically used. By comparing the biomarkers provided by these tests with typical ranges of values, a general idea of the health of the individual from which the blood was tested, can be determined. A great many conditions and illnesses cannot be determined with the basic tests. The primary markers indicating the condition or illness are not tested for, and therefore their values are not present. The primary markers that can be used to determine condition are often not tested for either because of their expense or the lack of sound rationale on the part of the clinician to pursue other tests from what the blood panel is or is not showing. Thus, they are often not run unless there is some specific reason to do so. Visible or detectable symptoms can certainly give reason to include additional blood tests. Additional diagnostic tests can support the confirmation of a specific condition or conditions. With many diseases it is prudent to begin treatment earlier rather than later, and waiting for detectible symptoms can be problematic for potential therapy. However, the more specific blood tests are not performed without some reason, due to their expense and the time involved. For this reason, many illnesses, which could be detected, are not determined in early stages.

It would be highly advantageous, therefore, to remedy the foregoing and other deficiencies inherent in the prior art.

An object of the present invention is to provide a method of determining a value of a biomarker not present in a conventional blood chemistry panel and CBC by employing the biomarkers that are present on the basic blood tests to computationally ascertain these additional values.

Another object of the present invention is to illuminate the need for additional testing through determination of specific biomarker values that can be garnered from the present method.

A further object of the present invention is early detection, diagnosis and quantification of medical conditions and illnesses.

### SUMMARY OF THE INVENTION

Briefly, to achieve the desired objects and advantages of the instant invention, a method for deriving a quantitative value for a specific biomarker of an individual includes the steps of providing a set of biomarkers from a blood sample of the individual, the set of biomarkers not including the specific biomarker. At least a portion of the set of biomarkers is manipulated with a precise numerical value to derive the quantitative value for the specific biomarker.

In a more specific aspect, the step of providing the set of biomarkers includes the step of providing the biomarkers from a blood chemistry panel and/or a complete blood count. At least a portion of the set of biomarkers can include adding adjusted marker values.

The desired objective of deriving a quantitative value of sub-markers from a specific primary marker found on a printed blood chemistry and CBC panel is achieved using standard biomarkers from the blood panel and calculating each of them with precise numerical values. This provides for the exact determination of specific sub-markers related to the available primary biomarker.

### DETAILED DESCRIPTION OF THE PREFERRED EMBODIMENTS

The present invention is a method of screening to determine the value of biomarkers indicating a condition or illness without the need for indiscriminate testing which would consume valuable testing resources and be very expensive. As an example, the ADA recommends screening of individuals with one or more diabetes risk factors at their next routine medical visit. The goal of screening is to identify asymptomatic individuals who are likely to have diabetes then perform separate diagnostics to establish the definitive diagnosis. Yet diabetes is typically diagnosed 5-9 years after the onset of the disease and often presents with one or more irreversible complications.

The method of the present invention includes obtaining and analyzing blood sample(s) in a conventional manner, such as through a commercial lab, to provide a set of biomarkers. The purpose of the method of the present invention is to use a set of biomarkers found through basic blood tests such as basic blood chemistry panels with complete blood counts to determine more exotic biomarkers without the time and expense of running specific tests for each biomarker. Once a set of typical biomarkers is assembled for a specific subject from the blood chemistry panel, a desired unknown biomarker can be determined through the use of biomarkers from the known set and adjusted marker values (AMV) averages of the extreme values of relevant basic diagnostic ranges, and then calculated with a precise numerical value (PNV). The calculations employed depend upon the unknown biomarker whose value is desired.

Each biomarker has a major diagnostic range that is the first indicator of health problems. These ranges are used to determine the adjusted marker values (AMV) which are mean numbers for each range that will be used to score an actual value from a blood report that is submitted for evaluation. The AMV is independent from the actual blood biomarker value. The PNVs are numerical constants based on a mathematical vectoring of the major systems of the body using biochemical, anatomical and mathematical data sets to create specific histochemically-adaptable formulas that allow for the accurate determination of a specifically desired biomarker.

The present invention provides a method capable of being used to diagnose a wide array of disease conditions with a minimum of a prior knowledge (ideally, with only the most basic of pre-existing medically relevant conditions, such as age and gender), thereby allowing for a fast and thorough health screening for use in high-throughput applications in which the search for specific health incongruities would be impractical (in other words, situations in which holistic health analyses are necessary to nonspecific health troubles). This method is also ideal as a type of “preprocessing” step in a more typical medical analysis, indicating both major problems and subtle imbalances requiring further attention as well as dispensing possible treatments and other therapeutic methods derived from the patient's basic health status. Examples of the present invention include the hormone cortisol and related cascade and the early detection of disease processes, Type 1 diabetes, pre-diabetes and Type 2 diabetes.

The biological significance of cortisol and its related submarkers correlate directly to the hypothalamic-pituitary-adrenal-thyroid (HPAT) axis, the quaternary glandular network that operates as the master control system for distributing and maintaining hormonal balance throughout the body. By assessing accurate cortisol levels through this diagnostic method, a descriptive picture of relevant submarkers akin to cortisol unfolds to provide specific characterization not only of the HPAT status but a variety of related indicators that can identify current health concerns as well as potential for oncoming disease implications.

An example of the method of the present invention focuses on cortisol and cortisol-related compounds and the consideration of their medical relevance to a variety of potential symptoms and/or conditions. The method of the present invention uses cortisol as only one of many primary biomarkers to evaluate the metabolism and establish an accurate summary of related analytes and biological systems. The method of the present invention can include other metabolic markers from the rest of the HPAT axis, which will then relate directly to the immune system at large, thereby maintaining the focus of the analysis on health irregularities.

The method of the present invention is intended to be used as an automated analysis routine designed as a custom “toolbox” (i.e. a set of prepackaged user-friendly operations set-up in the Mat LAB format). This toolbox is designed primarily to assess data found on a typical blood chemistry panel (as an example a Chem-25, Chem-20 or combination thereof), if possible with a lipid panel (measuring triglycerides, cholesterol and low- and high-density lipoproteins) and a complete blood count (red blood cell count and associated parameters, platelets, white blood cells, neutrophils, basophils, eosinophils, lymphocytes and monocytes). Due to pre-existing business practices, “typical” blood panel ranges will differ slightly depending upon which private company performs the screening. Panels tend to include most of the same quantifications such as blood urea nitrogen content, albumin concentration, glucose concentration, and roughly two dozen additional markers. It is also beneficial to include any and all additional information that the present method is capable of accommodating, if available.

The first step in the automated analysis, once the known variables are entered into the initial Mat LAB or program matrix, is the quantification of all biomarkers that can be derived from the measured biomarkers. There are over 70 blood panel variables that can be included in the diagnostic operation of the present invention that are not included in a typical blood chemistry panel (gastrin, ammonia, testosterone, and many others) which can improve and increase the end results of the analysis. If these variables are not present by way of the lab test almost all of them can be derived in a mathematical manner from the more common variables. These derivations are based upon a collection of Adjusted Marker Values (AMV's, essentially averages of the extreme values of relevant basic diagnostic ranges). Precise Numerical Values or PNV's are then used to perform linear mathematical operations with respect to the existing blood panel variables.

The function of PNV's is relatively easy to understand; a series of linear operations are performed which are combinations of pre-existing blood panel variables, ANV's, and PNV's. It should be noted here that most biomarker derivations will have over a dozen separate mathematical steps. The derivations are performed in this manner to continuously monitor the metabolic conditions under which the biomarkers operate and to assess relative norms in the patient. It is interesting to note that the manner in which the linear calculations are carried out is similar to the functioning of neural networks in which several input variables (in this case, the blood panel in question) undergo a set type of automatic linear calculations (metabolic corrections) and are then combined into some end result (the panel variable being defined).

The derivation program functions by performing every calculation that can be performed. This requires repeating the procedure enough times to take into account the derivation of numerical values that did not exist in previous equations. On future iterations, new variables might be derived by their own equations, and subsequently, other derivations which use these new variables can be performed.

Evaluation trials were performed to determine the accuracy of the automated versions of these algorithmic derivations. This was done by testing various blood panels with known biomarker quantities which could also be derived from other biomarker quantities on the same blood panel. The known biomarkers were eliminated from the panel, new values were calculated by the program and the percent difference between the known and derived quantities were calculated.

In an example of the method of the present invention, every blood panel variable is entered into the data matrix, or estimated based on other variables, each variable is then evaluated in terms of both a basic acceptable range and a series of secondary ranges based on Biological Range Variances (BRV's), determined in the same manner as the PNV values were determined (i.e. through ad hoc derivations performed over the course of practical application). The first basic range is the most fundamental factor determining whether or not a blood panel variable indicates a health irregularity; while these ranges are based mostly on commonly accepted ranges in medical tests, they have been modified to take into account differences in the manner in which various entities perform their blood screenings. Also, the basic ranges are usually decreased in overall size, making them more sensitive to major variations. For example, while the most commonly accepted diagnostic range for the variable amylase is 30-220 units, the algorithm being constructed will use a range closer to 50-200 units.

The BRV-based ranges are similar to the primary diagnostic ranges, though they are built to assess variables which lie within their respective primary diagnostic ranges. This assessment is based on which conditions are indicated (albeit in a non-definitive manner) by the variable that lies within a certain BRV-based range. An example of BRV-based range series for cortisol can be shown in an abbreviated form as follows.
Cortisol a.m. measurement (6-28 μg/dl), likeliest conditions in italics
6-10 Addison's disease, adrenal microadenoma
10-13 Addison's disease, hypopitultarism, microadenoma
13-15 adrenal insufficiency, stress
15-24 normal
24-28 pituitary dependant hyperadrenocorticism, (Cushing's analog), congenital adrenal hyperplasia
The BRV ranges alone allows the operator of the algorithm to quickly assess conditions indicated by the blood panel through the evaluation of these more subtle indicators. For example, if the condition of hypopituitarism is being evaluated by the program, a morning cortisol value of 12.6 would, according to the above BRV values, support evidence that this condition is indeed present.

At this stage, one can think of the algorithm as having fully deconstructed the data set into the most basic components suitable for the method of the present invention. These basic pieces of information are then evaluated in terms of which health problems are most strongly indicated in much the same way as unknown blood panel variables are derived; in other words, because it is possible to evaluate a health condition through BBID, this health condition will be assessed accurately. For example, gastrointestinal inflammation is evaluated by assessing all of the markers which have BRV ranges indicative of the condition such as the number of lymphocytes; if the lymphocyte BRV range indicates the presence of gastrointestinal inflammation, the practitioner has a probable target for diagnosis. Each condition will as a result of this evaluation process, be given a probability based on how many variables indicated the condition and how important each variable could be.

It is believed that the spreadsheets can function in the same manner as Mat LAB matrices, and that macros can be written to translate and interpret values between separate spreadsheets in the same manner as the evaluation described previously is performed. Finally, the BBID analysis itself can be potentially enhanced by applying novel chemometric techniques to the BRV range results. Because these results are the fundamental building blocks of the technique itself and play a supportive role in the analyses, it is likely that data mining techniques could be applied to these results, thereby deriving additional biomarkers and relationships between range values more subtle than have been determined by practical application. The method of the present invention can be potentially improved by the construction of Bayesian networks, which are themselves neural networks modified to use Bayesian statistical modeling. Bayesian neural networks are designed and constructed based on the formation of self-contained modeling algorithms and are usually designed so that they can “teach” themselves by the introduction of training sets. In this manner, the relationships between BRV range results, actual blood panel variables, and the underlying health conditions associated with the overall blood panel can be potentially targeted more specifically. As has been mentioned previously, the automated BBID technique can be mathematically thought of as a neural network in and of itself, as such a network would produce results based on calculation series similar to that described earlier in the variable derivations and BRV determinations; this makes the application of a self teaching Bayesian network thematically consistent and compelling.

Various changes and modifications to the embodiments herein chosen for purposes of illustration will readily occur to those skilled in the art. To the extent that such modifications and variations do not depart from the spirit of the invention, they are intended to be included within the scope thereof, which is assessed only by a fair interpretation of the following claims.

Having fully described the invention in such clear and concise terms as to enable those skilled in the art to understand and practice the same, the invention claimed is:

1. A method for deriving a quantitative value for a specific biomarker of an individual comprising the steps of:
providing a set of biomarkers from a blood sample of the individual, the set of biomarkers not including the specific biomarker; and
manipulating at least a portion of the set of biomarkers with a precise numerical value to derive the quantitative value for the specific biomarker.

2. A method as claimed in claim 1 wherein the step of providing the set of biomarkers includes the step of providing a blood chemistry panel.

3. A method as claimed in claim 2 wherein the step of providing the set of biomarkers includes the step of providing a complete blood count.

4. A method as claimed in claim 1 wherein the step of manipulating at least a portion of the set of biomarkers includes adding adjusted marker values.

5. A method for deriving a quantitative value for a specific biomarker of an individual comprising the steps of:
providing a set of biomarkers from a blood sample of the individual, the set of biomarkers not including the specific biomarker;
providing adjusted marker values for biomarkers not present in the set of biomarkers; and
using algorithmic manipulating of at least a portion of the set of biomarkers and the adjusted marker values with a precise numerical value to derive the quantitative value for the specific biomarker.

6. A method as claimed in claim 5 wherein the step of providing the set of biomarkers includes the step of providing a basic blood chemistry panel.

7. A method as claimed in claim 6 wherein the step of providing the set of biomarkers includes the step of providing a complete blood count.
